(19) United States
(12) Patent Application Publication
(10) Pub. No.: US 2018/0226141 A1
(43) Pub. Date: Aug. 9, 2018

(54) PATIENT COORDINATION SYSTEM AND METHOD

(71) Applicant: Arizona Board of Regents on Behalf of the University of Arizona, Tucson, AZ (US)

(72) Inventors: Marvin J. Slepian, Tucson, AZ (US); Fuad Rahman, Santa Clara, CA (US)

(21) Appl. No.: 15/746,762

(22) PCT Filed: Jul. 20, 2016
(86) PCT No.: PCT/US16/43178

§ 371 Date: Jan. 22, 2018

Related U.S. Application Data
(60) Provisional application No. 62/194,945, filed on Jul. 21, 2015.

(51) Int. Cl. G16H 10/60; G16H 80/00; G16H 40/20

(52) U.S. Cl. CPC G16H 10/60; G16H 40/20; G16H 80/00

(57) ABSTRACT

A patient coordination system, method and software product receives configuration of a hospital within a server. A hospital model is determined based upon the configuration. Location of each patient within the hospital is received. A course through hospital for each patient is received. Status for each patient is received from independently run hospital services. Progress of each patient through the corresponding course is determined and a dashboard showing the hospital model with spatial indication of the progress for each patient is generated.

### RELATED APPLICATIONS

This application claim priority to U.S. Patent Application Ser. No. 62/194,945, titled “Patient Coordination System and Method”, filed Jul. 21, 2015, and incorporated herein in its entirety by reference.

### BACKGROUND

The world population is ever increasing and the population is also aging as improved medical care allows people to live longer. Older people have greater healthcare needs and have more chronic diseases. Thus the number of patients being admitted into hospital is continually increasing. The increasing and aging population places an ever growing burden on the health system, and, in particular, on hospitals. Healthcare cost is directly related to the number of days each patient spends in the hospital. To assuage this increased healthcare burden, the number of available beds for newly admitted patients must increase, preferably without increasing healthcare costs. To accomplish this, the length of patient stay within the hospital is reduced, requiring that each patient be discharged from the hospital at the earliest opportunity. Any patient that occupies a bed longer than necessary is wasting hospital resources, increasing healthcare costs unnecessarily, and preventing admission of another patient. Further, unnecessary and prolonged hospitalization subjects patients to increasing debilitation form lack of activity, the risk of nosocomial infection and the possibility of medical error.

In the past, healthcare was focused largely on the relationship between the patient and the doctor. Today, healthcare has been broken up into distinct elements. These elements include: (a) personal medical care provided by the doctor or medical team, (b) physical therapy/occupational therapy that monitors and attempts to diagnose, facilitate and ensure patient mobility, (c) physical medicine and rehabilitation that ensures a patient obtains appropriate rehabilitation, with the goals of restored strength, movement and overall mobility and the like, (d) acceptability and availability of pharmacy that ensures a patient obtains prescribed medication(s), (e) psychological care that ensures that a patient is ready to leave the hospital for home or an intermediate step down type of facility, and (f) placement and social work services that ensures the patient has an adequate out-of-hospital domicile or living facility available, (g) end-of-life and hospice care, (h) ethics considerations regarding advanced treatment course, and (i) economics that ensure best available cost options selected for the patient.

As an example problem of today, a patient's progress through the hospital sometimes physically stops because there is no transport to take the patient from one department to another. Then, as transport staff become available, the order of patient movement occurs randomly as the transport department attempts to catch up by moving the more ready patient first. Consider for example a “Mr. Smith”, who is going to be in the hospital for another two weeks, but because he is ready, transport takes him before they take a “Mrs. Jones” awaiting tests that processes overnight. Therefore, the start of Mrs. Jones test is delayed such that her results are not available for her discharge the next morning and she remains in hospital an extra day.

For a patient to be discharged from the hospital, the patient must satisfy certain discharge or “exit” criteria, at a minimum. The patient must (a) pass certain medical tests at a satisfactory level, i.e. be ruled out for certain diagnoses or demonstrate lab or other criteria of improvement, (b) have necessary prescriptions written, authorized and/or fulfilled, (c) demonstrate adequate ambulation and self-care capability, i.e. if adequate thereby qualifying for unencumbered discharge or if inadequate requiring placement in a subsequent “step-down” care facility—e.g. a skilled nursing facility (SNF), inpatient rehab facility, or hospice, have necessary placement arranged, and (d) have information on follow-up care visits, outpatient procedures, labs or rehabilitation sessions. Currently, there is little coordination to ensure the patient has fulfilled each of these areas. Typically, a doctor and/or the medical team visits the patient during ‘rounds’ and determines, based upon information provided at the location of the patient—i.e. with info at the bedside, nursing unit and chart/EMR, whether or not to discharge the patient. The doctor therefore visits (rounds on) each patient, whether they are ready or not for discharge; thus, patients ready for discharge are kept waiting and occupy valuable hospital resources unnecessarily. Where the patient has not yet met all exit criteria, the doctor cannot discharge the patient. Further, the doctor/medical team may deem the patient ready for discharge only to put in orders and find out subsequently that the orders are not fulfilled and the patient is not discharged because an exit criteria component—e.g. availability of a bed at a rehab facility did not occur that day. Further, where the patient completes necessary tests and becomes ready for discharge after the doctor rounds, the discharge of the patient is often delayed until the next day, since the doctor typically rounds only once per day and is not presently, typically, informed “online” in “real time” as to the evolution of these events.

As another example of problems facing patient discharge, thirty to forty percent of hospital readmissions occur because more than sixty percent of patients leave the hospital without adequate information about drugs prescribed to them. Thus, the patient goes home; but due to lack of adequate information he or she fails to take prescribed medications, conditions recur and/or flair, and thereby the patient ends up returning to the hospital. These readmissions significantly increase the burden upon the hospital and increase overall healthcare costs.

### SUMMARY

In one embodiment, a patient coordination method receives configuration of a hospital within a server. A hospital model is determined based upon the configuration. Location of each patient within the hospital is received. A course through hospital for each patient is received. Status for each patient is received from independently run hospital services. Progress of each patient through the corresponding course is determined and a dashboard showing the hospital model with spatial indication of the progress for each patient is generated.

In another embodiment, a patient coordination system includes a processor, a memory communicatively coupled with the processor, an interface, communicatively coupled with the processor, capable of receiving status information of a plurality of patients of a hospital, and a patient status tracking algorithm. The patient status tracking algorithm, implemented as machine readable instructions stored within the memory and executed by the processor, is capable of: receiving configuration of a hospital; determine a hospital model based upon the configuration; receiving location of each patient within the hospital; receiving a course through hospital for each patient; receiving status for each patient from independently run hospital services; determining progress of each patient through the corresponding course; and generating a dashboard showing the hospital model with spatial indication of the progress for each patient.

### BRIEF DESCRIPTION OF THE FIGURES

FIG. 1 shows one exemplary patient coordination system, in an embodiment.

FIG. 2 illustrates creation of the hospital model, in an embodiment.

FIG. 3 shows a blueprint of the hospital of FIG. 1 being used to generate the hospital model, in an embodiment.

FIG. 4 shows exemplary patient status for the patient of FIG. 1 illustrating a plurality of discharge criteria, in an embodiment.

FIG. 5 shows one exemplary dashboard that simultaneously displays discharge readiness for a plurality of patients, in an embodiment.

FIGS. 6A-6C show exemplary single patient dashboards illustrating changes in discharge readiness of the patient of FIG. 1, in an embodiment.

FIG. 7A shows the hospital overview patient discharge dashboard of FIG. 1 in further exemplary detail, in an embodiment.

FIG. 7B shows one exemplary patient display that is generated when one sphere of the dashboard of FIGS. 1 and 7A is selected, in an embodiment.

FIG. 7C shows one exemplary worksheet that is displayed when the laboratory test results indicator of FIG. 7B is selected, in an embodiment.

FIG. 8 shows the hospital services of FIG. 1 in a further embodiment.

FIG. 9 is a schematic illustrating exemplary movement of the patient of FIG. 1 through the hospital, in an embodiment.

FIG. 10A is a Gantt chart illustrating exemplary timing of the hospital services of FIG. 1 for the patient within hospital, in an embodiment.

FIG. 10B is a Critical Path Chart illustrating exemplary timing of the hospital services of FIG. 1 for the patient within hospital, in an embodiment.

FIG. 11 shows one exemplary zoomed view of a portion of the hospital of FIG. 1 showing a status of each patient, in an embodiment.

FIG. 12 is a flowchart illustrating one exemplary patient coordination method, in an embodiment.

### DETAILED DESCRIPTION OF THE EMBODIMENTS

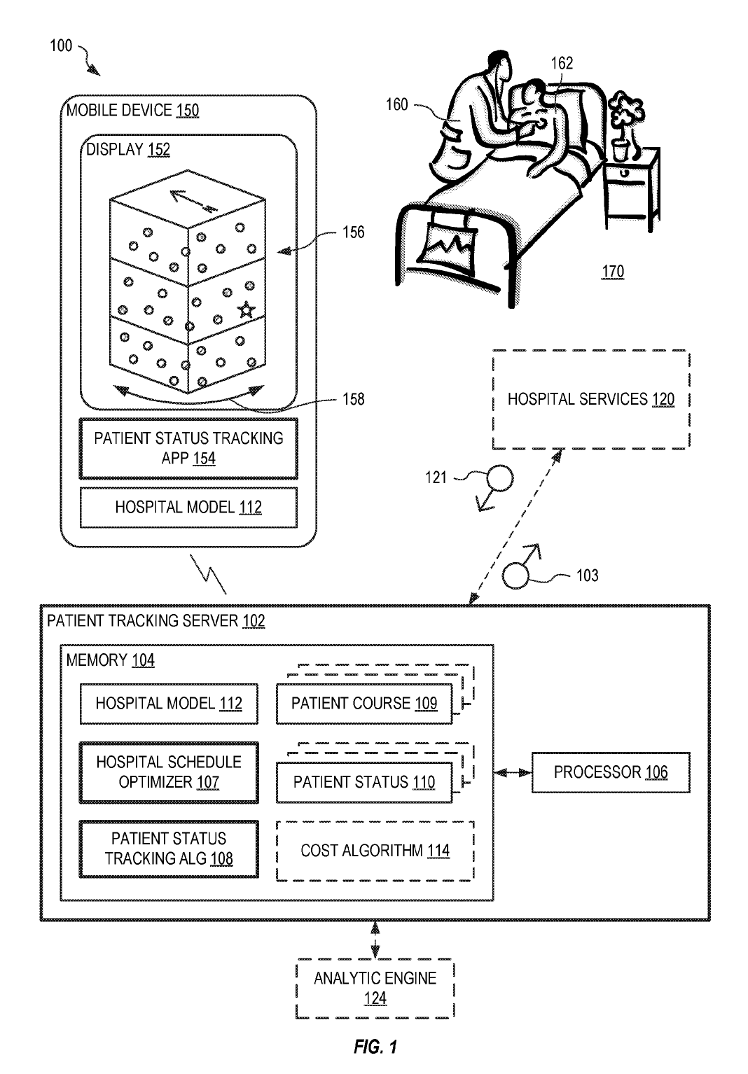

FIG. 1 shows one exemplary patient coordination system 100. System 100 includes a patient tracking server 102 and a mobile device 150. Although mobile device 150 represents a preferred embodiment, information and interaction with mobile device 150 may also occur using a screen on any accessible digital device, such as one or more of a fixed terminal at patient bedside, a nursing station, a computer on wheels (COW), a tablet, a smartwatch, and a smartphone, or the like. Server 102 is a networked computer and includes a memory 104 and a processor 106. Memory 104 is shown storing a patient status tracking algorithm 108 that has machine readable instructions that are executable by processor 106 to provide functionality described herein below for server 102.

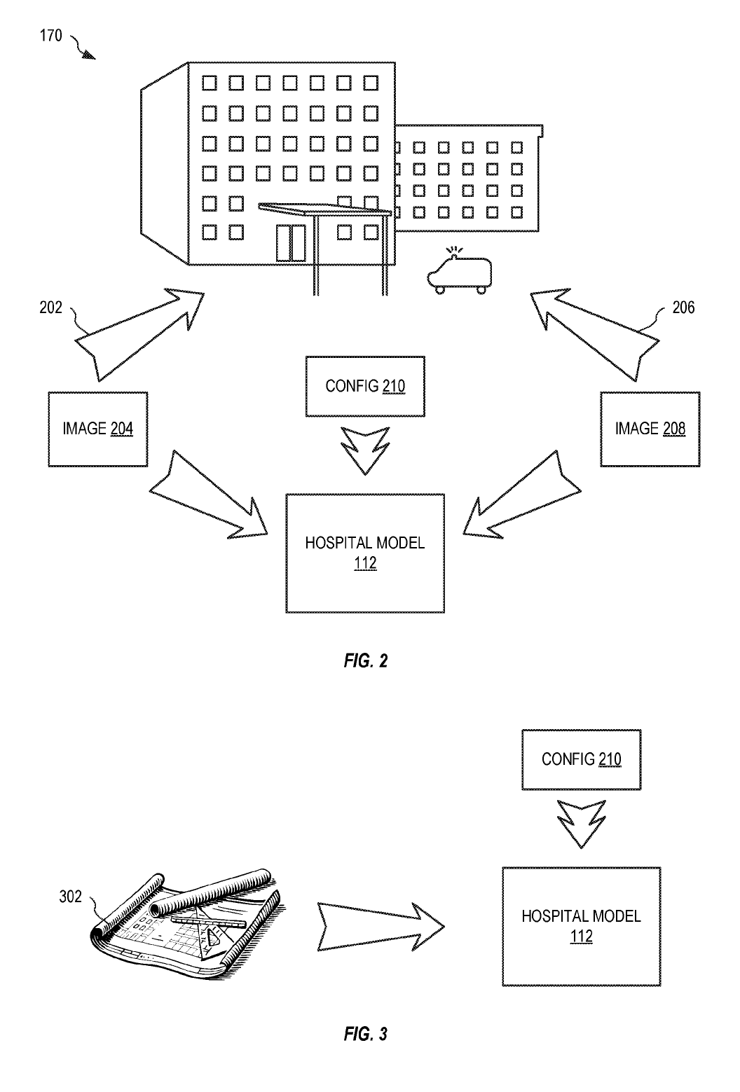

Memory 104 also stores a hospital model 112 that defines a structure and location of patients (e.g., patient 162) within hospital 170. FIG. 2 shows exemplary creation of hospital model 112; specifically FIG. 2 shows capture of two images external to, and of, hospital 170 used to create hospital model 112 and configure system 100 for use within hospital 170. Referring again to FIG. 1 and the FIG. 2 example, patient tracking server 102 operates to generate hospital model 112 from images 204 and 208 such that hospital model 112 has an appropriate number of floors, appropriate shape (e.g., where hospital 170 has one wing that protrudes from a main tower), and so on. An installer may then enter a configuration 210 defining the portion of hospital 170 that is used for medicine, including: a number of floors, a number of wards, and a number of beds per ward, for example (and more or fewer configurations exist without departing from the scope hereof). Algorithm 108 then automatically generates hospital model 112 based upon images 204, 208 and the entered configuration. Alternatively, where images are not available, algorithm 108 may generate a generic structure for containing the defined number of floors, beds, and wards based upon configuration 210. FIG. 3 shows a blueprint 302 of hospital 170 being used to generate hospital model 112 in an alternate embodiment, optionally together with configuration 210.

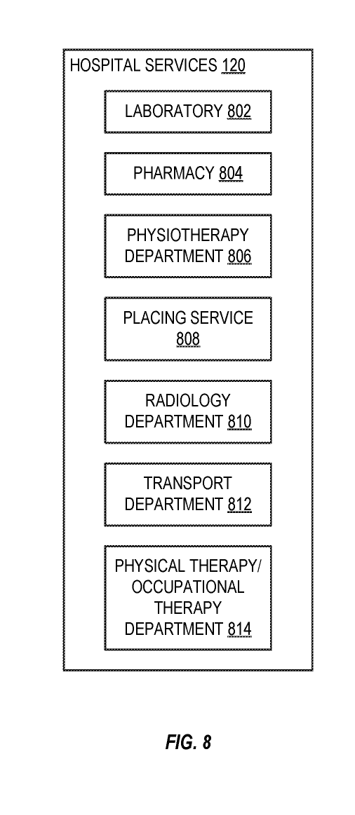

Server 102 is also configured to request 103 services for patient 162 from hospital services 120 and may receive information 121 from hospital services 120 as to when the requested services may be provided to patient 162. Hospital services 120 represents services typically found in a hospital, including pharmacy services, physiotherapy services, rehabilitation services, psychological services, radiology services, patient monitoring sensors, or other data generating and providing means, and so on. See FIG. 8 for further exemplary details of hospital services 120. Server 102 may include an interface for interactively receiving input from nurses and doctors as to progress of patient 162 with each of these services. Server 102 may also include an interface for receiving patient status information directly from other computer systems used by the hospital services 120.

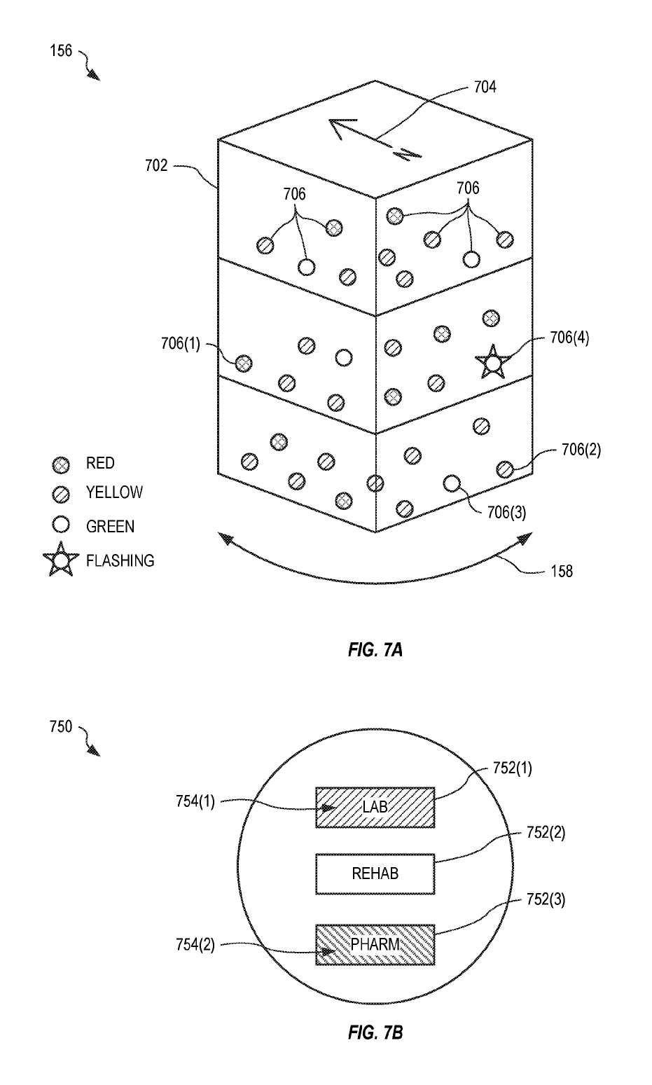

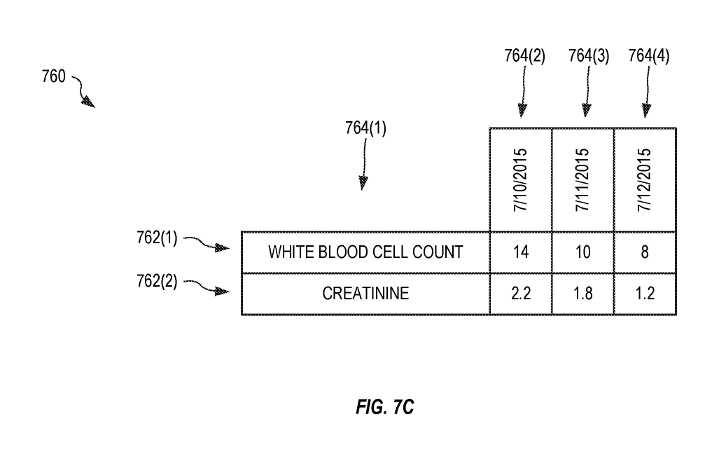

In the preferred embodiment, server 102 is wirelessly communicatively coupled with mobile device 150 which is carried by a doctor 160 for example. Doctor 160 may represent one or more of a doctor, a nurse, a case manager, a pharmacist, a social worker, a physical and occupational therapist, a dietician, a hospital administrator, a chaplain, a counselor, an ethicist and other patient-related health care personnel. Mobile device 150 represents one of a smart phone, a tablet device, a personal digital assistant (PDA), and other portable communication devices with similar capability. As noted above, mobile device 150 may also be implemented as a fixed or somewhat mobile interface, e.g., a fixed terminal at patient bedside, a nursing station, and a COW, without departing from the scope hereof. Mobile device 150 includes a memory and a processor, not shown for clarity of illustration, which stores and executes a patient tracking app 154. Mobile device 150 also includes a display 152 that is illustratively shown displaying a hospital overview patient discharge dashboard 156, which is described in further detail with respect to FIG. 7A.

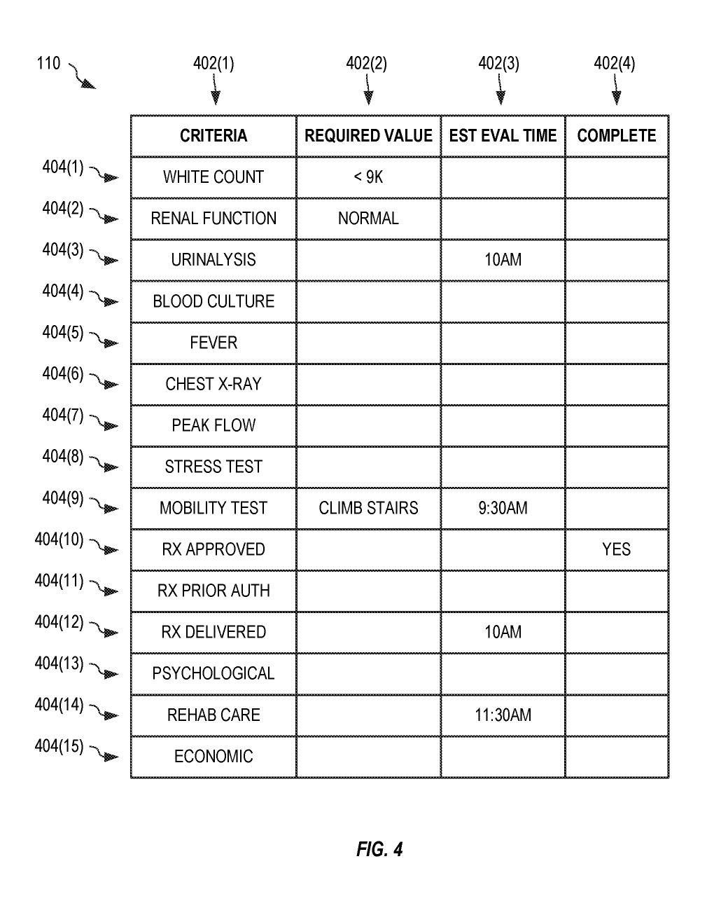

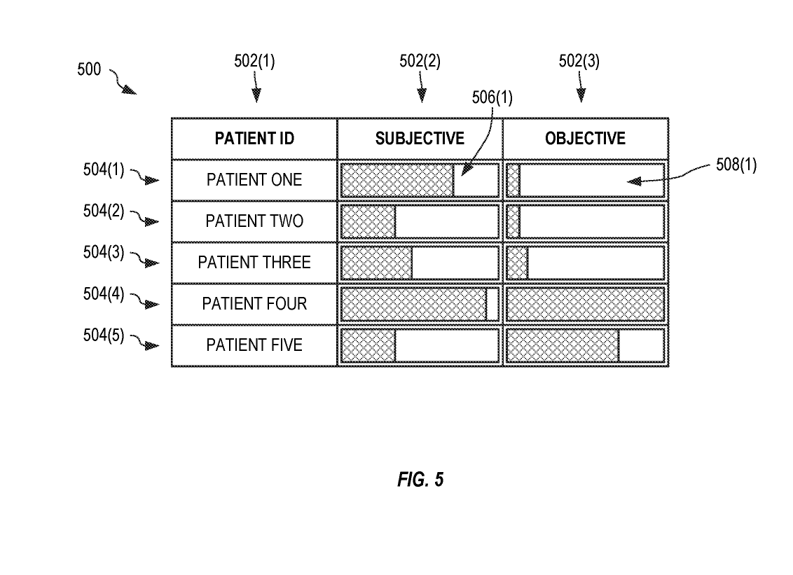

Memory 104 also stores, for each tracked patient (e.g., patient 162), a patient status 110 that defines the readiness of the patient for discharge. Patient Status 110 is shown in further detail in FIG. 4.

FIG. 4 shows exemplary patient status 110 for patient 162 illustrating a plurality of discharge criteria 404. These “discharge” criteria are the essential variables “distilled” down form the myriad of pieces of information/exam findings/lab results/imaging info and the like that are the critical determinants or guideposts being monitored to determine the safety and suitability for a patient to be discharged from an acute care facility. Patient status 110 is a data structure that includes four columns 402(1)-(4) and fifteen data rows, each row storing one criterion 404 that may need to be met before patient 162 is ready for discharge from hospital 170. As shown in name column 402(1) of FIG. 4, discharge criteria 404(1)-(15) respectively represent: white count within defined limits, renal function within limits, urine analysis within limits, blood culture within limits, fever below a given temperature, chest x-ray clear, peak flow, stress test passed, mobility test passed, prescribed drugs approved, prescribed drugs prior authorized if required, prescribed drugs delivered to patient 162, patient psychologically ready to leave hospital, rehabilitation care established, discharge of patient 162 economically viable. Column 402(2), for each criteria 404, defines an acceptable threshold or range for the defined criterion. For example, a white count less than nine is defined for the white count criterion 404(1).

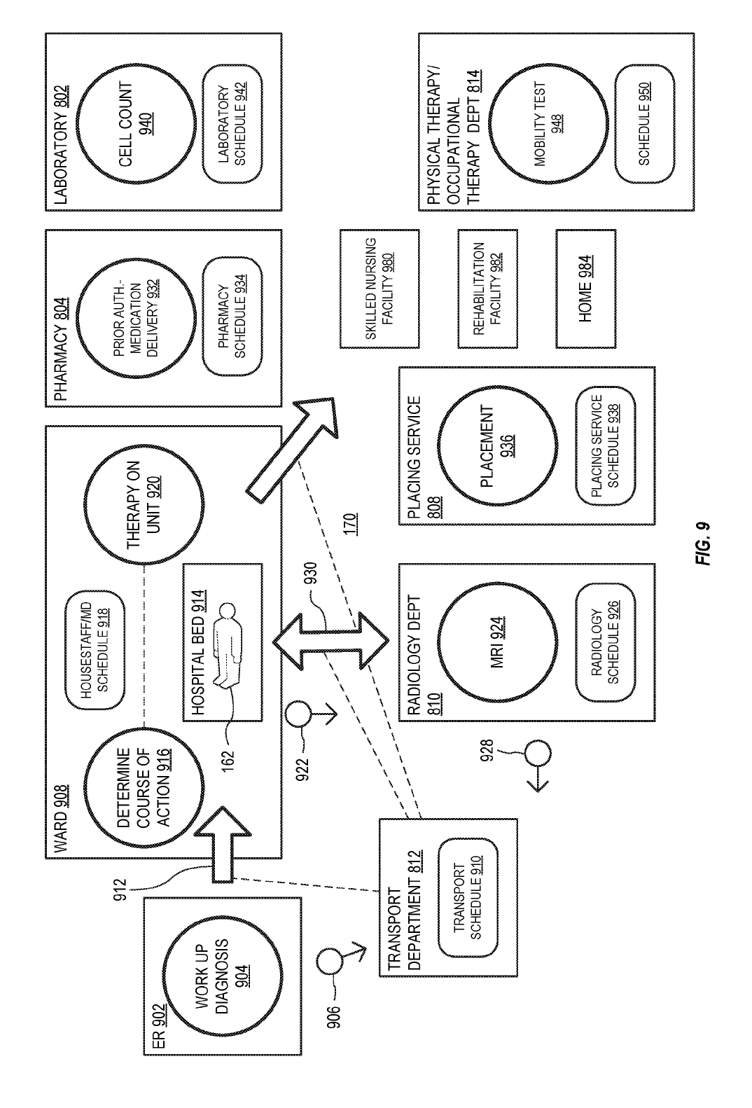

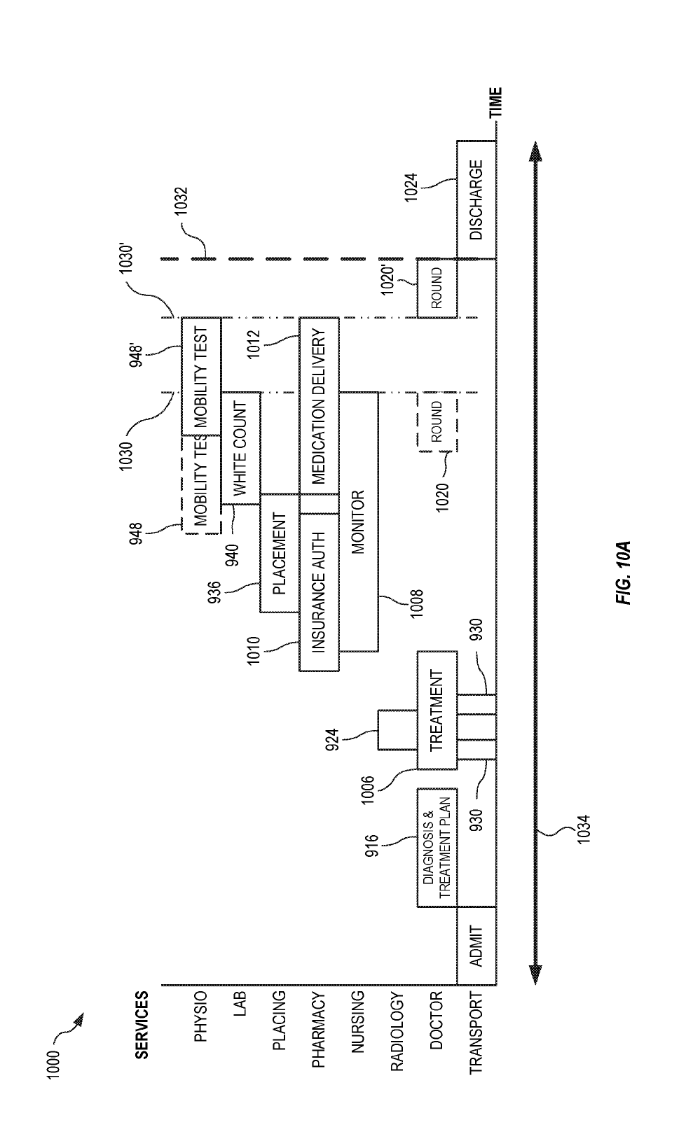

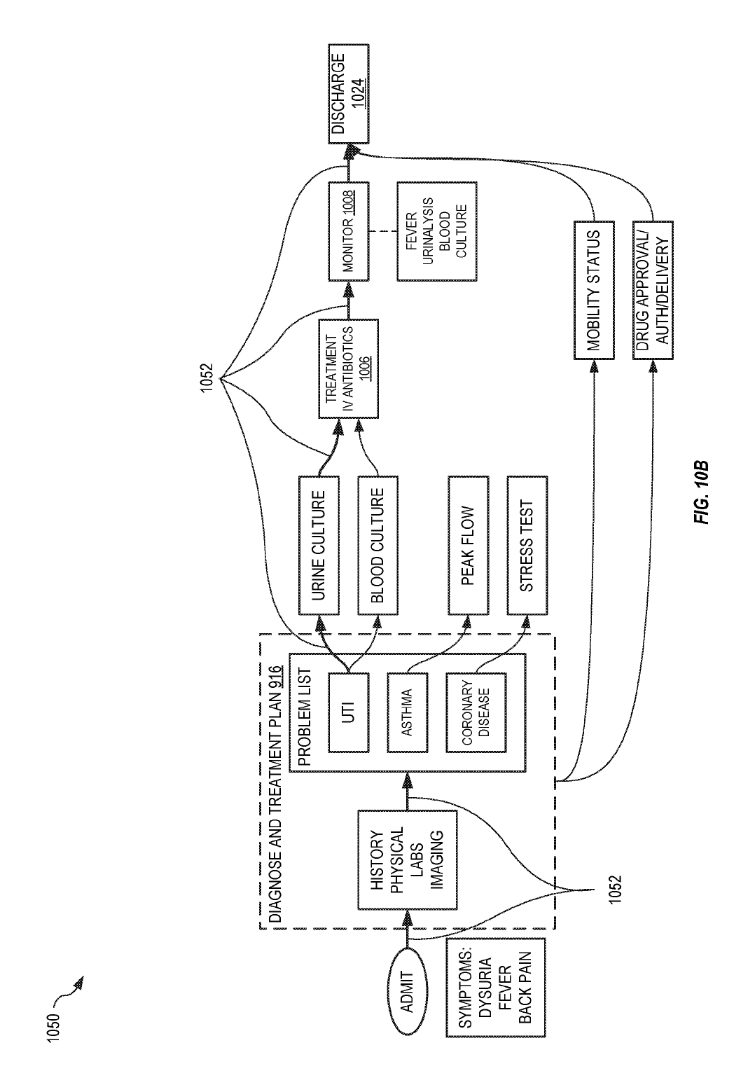

Column 402(3) stores an estimated evaluation time that indicates when results, or an evaluation of the corresponding criteria, may be expected. In the example of FIG. 4, the urinalysis criteria 404(3) is expected to complete at ten o'clock in the morning. The estimated evaluation times of column 402(3) may be provided by specific departments within hospital services 120 based upon available scheduling of equipment, staff, and so on. In one example of operation, patient status tracking algorithm 108 receives an estimated completion time for a stress test on patient 162 from hospital services 120 and stores the estimated time within patient status 110. See FIGS. 9 and 10 and accompanying description for further details of how scheduling of tests may be affected by system 100.

Column 402(4) stores an indication as to whether the corresponding criterion 404 has been met. For example, if algorithm 108 receives results of a white blood count in a sample taken from patient 162 and determines that the count meets the required value specified in column 402(1) (e.g., less than nine thousand), algorithm 108 marks (e.g., sets to “YES”) column 402(4) of criterion 404(1) to indicate that the white count criterion has been met. Algorithm 108 automatically updates patient status 110 as information is received from hospital services 120 such that patient status 110 is up-to-date.

As shown in FIG. 4, not all criteria 404 are required for patient discharge. Where patient 162 is being treated for a urinary infection, for example, chest x-rays criterion 404(7), Peak flow criteria 404(7) and stress test criterion 404(8) are is not required for discharge of patient 162. Based upon relevance to patient 162, system 100 presents a “distilled” endpoint list that shows information relevant to the patient, and may include information on secondary problems being tracked for patient 162. For example, although patient 162 is admitted with a urinary tract infection, where patient 162 also has a history of asthma with mild exacerbation and shortness of breath, a stress test may have been performed during the admission to rule out a cardiac component of the presenting symptoms. Based upon diagnosis and treatment of patient 162, relevant criteria 404 may be defined as required for discharge of patient 162. Further, system 100 may display certain less relevant results when they indicate abnormal results.

Continuing with the example where patient 162 has a urinary infection, given the patient's symptoms (e.g., back pain, cloudy urine, etc.), the doctor may diagnose and treat a urinary infection, defining criteria 404(1) requiring a white blood cell count value of less than nine thousand before the patient is allowed to go home. For example, the doctor may prescribe tests such as white blood cell count, a blood culture and a urinalysis. Other criteria, such as peak flow, chest x-ray, etc., that are not relevant to the diagnosis and treatment, are not defined and therefore do not prevent release of patient 162. Thus, once results for the white count and urine analysis return to normal, patient 162 may be released.

In another example, patient 162 is readmitted to hospital with recurring symptoms that have not been successfully treated using a generic drug, the doctor may prescribe a newer drug. However, for this newer drug, once it is approved, it may require prior authorization by the patient's insurance company. An insurance company may automatically authorize generic drugs, whereas for newer, more expensive, drugs the insurance company may require that the specific circumstances of the patient are evaluated prior to authorization. Therefore, once the drug is prescribed and approved, and without waiting until the patient is about to be discharged, prior authorization for the drug may be requested. Since, such prior authorization may take twenty-four to forty-eight hours to obtain, the earlier this process is started, the less likely it will delay discharge of the patient.

Thus, the use of patient status 110 and the defined criteria 404 required for discharge allows better planning and coordination of the required care and tests such that discharge of the patient is not delayed unnecessarily.

FIG. 5 shows one exemplary dashboard 500 that simultaneously displays discharge readiness for a plurality of patients. Dashboard 500 may be displayed upon display 152 of mobile device 150 for example to provide a discharge readiness indication for all patients assigned to doctor 160. In dashboard 500, each data row 504(1)-(5) represents one patient (e.g., patient 162) of doctor 160, where column 502(1) of each row 504 identifies the patient (e.g., using a unique number and/or the name of the patient). For each patient (e.g., row 504), a subjective column 502(2) of dashboard 500 indicates completeness of all subjective criteria (e.g., psychological status criterion 404(13) of patient status 110, rehabilitation care criterion 404(14), and economic circumstances criterion 404(15)) and includes a bar graph 506 to indicate overall completeness of subjective criteria. For each patient (e.g., row 504), an objective column 502(3) of dashboard 500 indicates completeness of all objective criteria (e.g., white count criterion 404(1), renal function criterion 404(2), urine analysis criterion 404(3), and so on, of patient status 110) and includes a bar graph 508 to indicate overall completeness of subjective criteria (column 502(2)). Each bar graph 506, 508, thereby provides rapid assimilation by doctor 160 of each patient's readiness for discharge. Further, for each bar graph 506, 508, the background color may further indicate readiness for discharge. For example, where the background of bar graph 506(1) is green, and the background of bar graph 508(1) is red, the doctor may easily assimilate that the objective criteria 404 are not going to allow patient one to go home.

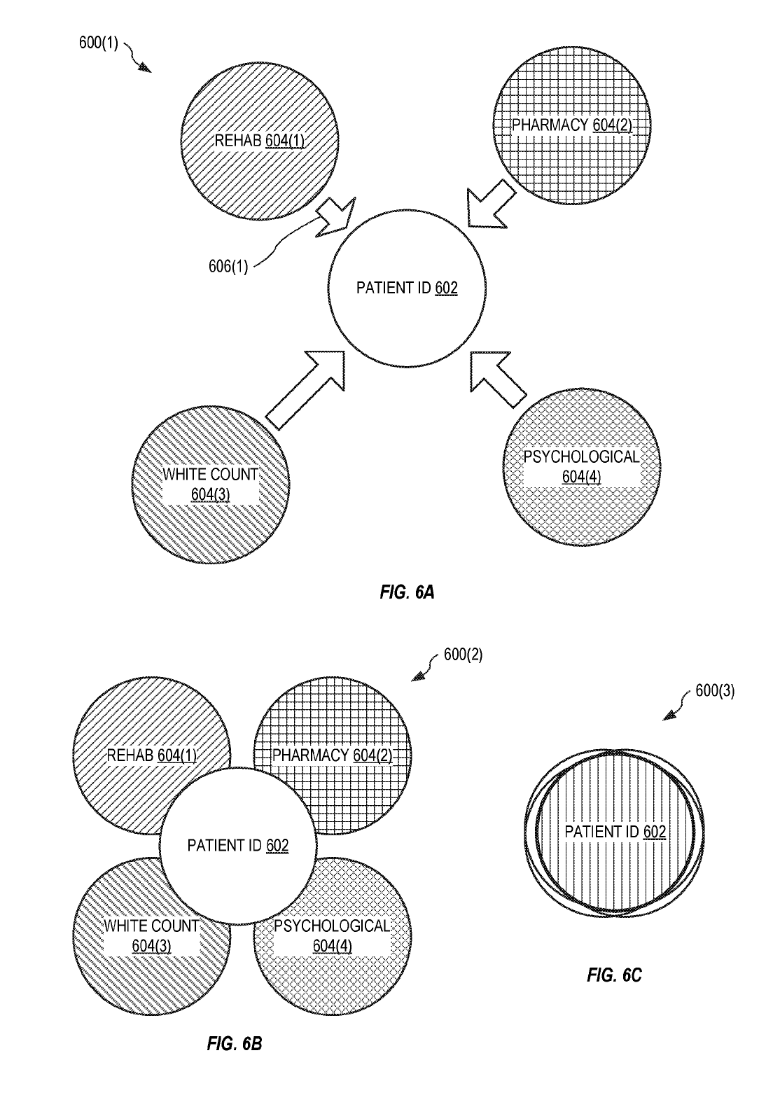

FIGS. 6A through 6C show exemplary single patient dashboards 600(1), 600(2), and 600(3), respectively, illustrating changes in discharge readiness of patient 162. Each dashboard 600 has a center circle 602 that identifies the patient (e.g., patient 162), and four criteria circles 604(1)-(4) that each correspond to one criteria required for discharge of patient 162. Fewer or more criteria circles 604 may be displayed depending upon the relevant criteria 404 for discharge of patient 162. In the example of FIGS. 6A, 6B, and 6C, circle 604(1) corresponds to rehab care criterion 404(14) of patient status 110; circle 604(2) corresponds to one or both of RX approved criterion 404(10) and RX delivered criterion 404(12) of patient status 110; circle 604(3) corresponds to white count criterion 404(1) of patient status 110; and circle 604(4) corresponds to psychological criterion 404(13) of patient status 110. Certain criteria 404 may be grouped together for display in a single circle 604 of dashboard 600, and non-relevant or non-important criteria 404 are not displayed at all. For example, where patient 162 has a urinary infection, chest x-ray criteria 404(7) is not relevant, and therefore not displayed. Patient 162 may have an EHR that includes hundreds of pieces of information, and display of all this information is not easily assimilated, since doctor 160 would need to search for information of interest. Dashboard 600, on the other hand, displays only information that is currently relevant to patient 162, thereby making this information more readily available and easily assimilated by doctor 160. For example, doctor 160 primarily needs to know how ready patient 162 is for discharge, and to be made aware of reasons why patient 162 is not ready for discharge such that further action may be taken if needed.

In the example of FIG. 6A, dashboard 600(1) indicates that patient 162 is not ready for discharge, since circles 604 are separated from center circle 602. As each criterion nears readiness, the corresponding circle 604 moves towards center circle 602 as indicate by arrows 606. Dashboard 600(2) of FIG. 6B shows patient 162 approaching readiness for discharge, and dashboard 600(3) of FIG. 6C shows patient 162 ready for discharge, since circles 604 are substantially co-located with center circle 602 which has changed color (e.g., to green from red) for faster assimilation by doctor 160. In particular, dashboard 600 allows doctor 160 to easily see the discharge readiness status of patient 162, and where patient 162 is not ready for discharge, doctor 162 may easily see which one (or more) criterion is preventing discharge based upon the distance of circle 604 from center circle 602.

FIG. 7A shows hospital overview patient discharge dashboard 156 of FIG. 1, in further exemplary detail. Dashboard 156 includes a wire frame 702 that represents structure of hospital 170 and a direction indicator 704 that provides orientation of wire frame 702. In the example of FIGS. 1 and 7, indicator 704 points north, thereby enabling doctor 160 to orient wire frame 702 relative to the earth (though any other orientation may be used, for example orientation to a local geographic structure like a mountain range). Wire frame 702 is not necessarily dimensionally proportional to hospital 170, but provides a reasonable representation that is easily understood by doctor 160. Arrows 158 allow doctor 160 to rotate wire frame 702 (and other components of dashboard 156) to allow alternate views thereof. For example, where a current view results in one patient being hidden, doctor 160 may rotate wire frame 702, using arrows 158, such that the patient is no longer hidden.

Within dashboard 156, patients are represented as spheres 706, spatially positioned within wire frame 702 based upon location of the patient within hospital 170, where the color of each sphere indicates discharge readiness. For example, a first patient is displayed as a red sphere 706(1) indicating that they are not close to being ready for discharge, a second patient is displayed as a yellow sphere 706(2) indicating that they are close to being ready for discharge, and a third patient is displayed as a green sphere 706(3) indicating that they are ready for discharge and awaiting doctor 160. A fourth patient is displayed as a flashing green sphere 706(4) indicating that they have been discharged. Thus, dashboard 156 provides an immediate overview of patient discharge within hospital 170.

FIG. 7B shows one exemplary patient display 750 that is generated when one sphere 706 of dashboard 156 is selected. For example, when doctor 160 taps on sphere 706(2) within dashboard 156, patient display 750 is generated and displayed on mobile device 150. Patient display 750 shows a more detailed overview patient 162 corresponding to sphere 706(2), illustrating that additional information is available for one or more area. As shown in the example of FIG. 7B, patient display 750 includes a laboratory test results indicator 752(1), a rehabilitation status indicator 752(2), and a pharmacy status indicator 752(3). A background color 754 may also be provided to indicate significance of each area with relevance to progress of patient 162 towards becoming ready for discharge. For example, where laboratory test results to not meet one or more criteria 404, laboratory test results indicator 752(1) may have a background color 754(1) of red. A background color of green may be used to indicate that laboratory tests results for patient 162 meet discharge criterial 404.

FIG. 7C shows one exemplary worksheet 760 that is displayed when laboratory test results indicator 752(1) is selected. Information within worksheet 760 may be displayed in other formats without departing from the scope hereof. For example, information may be displayed in graphical form where appropriate.

Worksheet 760 has four columns 764(1)-(4), where a first column indicates the name of the test, and each of columns 764(2)-(4) indicated a test result for a particular date. Rows 762(1) and (2) indicate specific test and their results. In particular, row 762(1) shows white blood cell count results, and row 762(2) shows creatinine results.

FIGS. 7A-C collectively show how further detail may be obtained from dashboard 156.

Optionally, dashboard 156 also shows a current location of doctor 160, based upon a current location determined by mobile device 150 for example. In one embodiment, location of each patient may be defined based upon assignment of the patient to a particular bed within hospital 170. In another embodiment, each patient has an attached (e.g., wrist band) tag that is locatable within hospital 170. For example, the tag may be an RFID tag that is identified and located within hospital 170. In another embodiment, each bed includes a locator that defines the location of the bed within hospital 170, wherein the patient is located based upon determined location of the bed. Similarly, the location of vital personnel particularly relevant to patient throughput and discharge may also be tracked and visualized on dashboard 156. Personnel to be tracked include: case manager, head nurse, unit clerk, pharmacist, physical therapy team, social worker and dietician/nutritionist, for example.

In one embodiment, analytic engine 124 collects video data from a plurality of cameras positioned within medical facilities (including hospital 170) and identifies people (e.g., patients, doctors, and staff) as they move around within the medical facilities. Healthcare analytic engine 124 thereby tracks patient 162 as he/she moves around these medical facilities to learn more of their behavior and of their current location within hospital 170. For example, analytic engine 124 may determine whether patient 162 is confused as to where to go as they arrive at or leave hospital 170. Analytic engine 124 may determine if they always arrive at medical facilities early, whether they move in a confident manner, and so on. By analyzing movement of people, analytic engine 124 may also provide information for optimizing building and pathway layout.

Although analytic engine 124 primarily monitors patients, analytic engine 124 may also monitor movement of staff within the medical facility to determine where their time is being spent. For example, where health staff members are critical for expediting patient discharge, or where certain staff members are delaying patient discharge, it may be useful to locate these staff members for communication purposes. Thus, by using analytical engine 124 to track staff members, communication delays may be avoided. By monitoring staff member locations, analytic engine 124 may also predict availability of staff members to complete subsequent tasks. For example, a social worker may be needed to sign-off on a first patient ready for discharge, but, that social worker may already be busy working with a second patient that is having problems finding rehabilitation accommodation. Since analytic engine 124 is aware of the social workers location and current activity, analytic engine 124 may predict when the social works will become available to attend the first patient, thereby allowing other tasks to be reschedule for optimal efficiency, as described below.

In one embodiment, each patient may have an arm band that provides electronic identification of the patient. For example, the arm band may include computer readable markings and/or computer readable wireless identification (e.g., RFID tags) capability. Armband readers positioned within specific locations of the hospital, such as particular departments, corridors, and so on, may identify proximity of the patient from the armband, thereby facilitating tracking of the patient through various hospital departments as prescribed tests are performed on the patient. Optionally, electronics within the arm band may be used to store certain information of the patient, such as results from performed tests, allowing a doctor to quickly retrieve these results when attending the patient.

Similarly tracking tags for patient and staff may also have active telemetry or radio broadcast means to telemeter or broadcast location and status/data of the wearer.

### Hospital Service Scheduling

FIG. 8 shows hospital services 120 of FIG. 1 in further exemplary detail. In the example of FIG. 8, hospital services 120 include a laboratory 802 for testing samples taken from patients, a pharmacy 804 for providing prescribed medication to patients, a physiotherapy department 806 that provides physiotherapy services to patients, a placing service 808 that places patients within rehabilitation centers, and a radiology department 810 that performs radiology services on patients.

FIG. 9 is a schematic illustrating exemplary movement of patient 162 through hospital 170. FIG. 10A is a Gantt chart 1000 illustrating exemplary timing of hospital services 120 for patient 162 within hospital 170. FIG. 10B is a critical path chart 1050 illustrating exemplary timing of hospital services 120 for patient 162 within hospital 170. FIGS. 9, 10A and 10B are best viewed together with the following description.

Gantt chart 1000 and critical path chart 1050 may be displayed on display 152 of mobile device 150, for example in response to doctor 160 selecting one patient from dashboard 156 by double clicking/tapping on a corresponding sphere 706. Gantt chart 1000 may also be displayed on other devices coupled with server 102, e.g., a nurse's station, a computer on wheels, and so on. Information in Gantt chart 1000 may also be displayed as a critical path chart without departing from the scope hereof. For example, in critical path chart form, actions and results that directly affect the patient discharge date and time may be more easily identified since a critical path 1052 is indicated.

In the example of FIGS. 9 and 10, patient 162 has visited ER 902 and ER staff has generated a work-up diagnosis 904 for patient 162. ER staff then sends a request 906 asking transport department 812 to move patient 162 to ward 908 of hospital 170. Transport department 812 has a transport schedule 910 that allocates transport staff members and equipment to each request. Based upon transport schedule 910, patient 162 is moved 912 from ER 902 to hospital bed 914 within ward 908.

Within ward 908, patient 162 is added to housestaff/MD schedule 918 and housestaff (e.g., doctor 160), based upon schedule 918, determines a diagnosis and treatment plan 916 for patient 162. The determined treatment plan is stored within server 102 as patient course 109 and defines actions to be taken for patient 162 while within hospital 170. In the example of FIGS. 9 and 10, patient course 109 defines a therapy on unit 920 to treat patient 162 and includes obtaining a MRI scan 924 from radiology department 810. Accordingly, housestaff within ward 908 send a request 922 asking radiology department 810 to schedule MRI 924 of patient 162. Radiology department 810 then sends a request 928 to transport department 812 requesting patient 162 be moved 930 to radiology department 810, and returned to ward 908, based upon radiology schedule 926. Accordingly, request 928 is added to transport schedule 910, and patient 162 is moved 930 radiology department 810 based upon transport schedule 910. MRI 924 is taken of patient 162 based upon radiology schedule 926, and patient 162 is returned to ward 908 based upon transport schedule 910.

Upon completing treatment 1006 (e.g., therapy on unit 920, MRI 924, and so on), patient 162 may be monitored by housestaff (e.g., nurses) within ward 908 for a period, indicated in FIG. 10A as monitor 1008. Assuming treatment 1006 was successful and patient 162 is recovering as expected, system 100 generates a predicted discharge time 1030 and preparation for discharge of patient 162 begins.

Analytic engine 124 continuously collects healthcare information from many locations including hospital 170, as described in Appendix A and Appendix B of U.S. Patent Application Ser. No. 62/194,945. Thus, by processing healthcare information collected within hospital 170 for patient 162, analytic engine 124 learns of discharge requirements for patient 162, and sends these requirements to server 102. Patient status tracking algorithm 108 thereby automatically updates patient status 110 regarding requirements for discharge of patient 162. For example, during rounds, doctor 160 may determine that patient 162 is “doing well” and say to patient 162 “if your white count is less than nine, you can go home tomorrow.” Analytic engine 124 uses natural language processing to understand this discharge requirement and sends the requirement to server 102, where algorithm 108 updates patient status 110 to add criterion 404, as described above. Similarly, as other services visit patient 162 to discuss discharge, server 102 learns of discharge requirements. Further, based upon the doctor's comments to patient 162, analytic engine 124 also learns and informs server 102 of the expected discharge time, this algorithm 108 may update predicted discharge time 1030. Discharge requirements for patient 162 may also be entered to system 100 directly by each hospital service 120, such as through a web interface or other means.

As time progresses, pharmacy 804 receives a prescription for medication for patient 162 and adds the prescription to a pharmacy schedule 934. Based upon schedule 934, pharmacy 804 initiates drug prior authorization and medication delivery 932 to patient 162, shown as insurance authorization 1010 and medication delivery 1012 within Gantt chart 1000. Concurrently with actions by pharmacy 804, placing service 808 adds placement 936 for patient 162 to placing service schedule 938. Based upon placing service schedule 938, placing service 808 determines placement 936 by booking patient 162 into one of a skilled nursing facility 980 and a rehabilitation facility 982, or verifies that patient 162 has adequate care at home 984.

Concurrently with actions by pharmacy 804 and placing service 808, laboratory 802 adds a blood test for patient 162 to laboratory schedule 942. Based upon laboratory schedule 942, laboratory 802 performs a white count 940 on a sample from patient 162 and delivers the result to system 100.

Concurrently with actions by pharmacy 804, placing service 808, and laboratory 802, physical therapy/occupational therapy department 814 adds a mobility test 948 for patient 162 to schedule 950. Based upon schedule 950, physical therapy/occupational therapy department 814 performs mobility test 948 on patient 162 to determine whether patient 162 is able to go home.

In the prior art, each hospital service 120 operates independently of other services, basing its actions upon its own schedule (e.g., laboratory 802 bases its action on laboratory schedule 942, pharmacy 804 bases its actions on pharmacy schedule 934, and so on). However, where any actions is delayed or rescheduled within that department, other departments are not aware of those changes. System 100 operates to optimize scheduling within each hospital service 120 to maximize hospital patient throughput as a whole. Thus, as any hospital service schedule (e.g., schedules 910, 918, 926, 934, 938, 942, and 950) is adjusted, that adjustment is sent to server 102, wherein a hospital schedule optimizer 107 recalculates the priority of each item within all other schedules of hospital services 120.

In the example of FIGS. 9 and 10, medication delivery 1012 is delayed (e.g., a specific drug has to be shipped from a supplier) and cannot complete until after predicted discharge time 1030. Accordingly, algorithm 108 adjusts predicted discharge time 1030, indicated as updated discharge time 1030′. Optimizer 107 then adjusts priority for patient 162 for other actions scheduled within other hospital services, since patient 162 is unable to be discharged until medication delivery 1012 completes. Accordingly schedule 950 is updated to allow physical therapy/occupational therapy department 814 to service another patient before visiting patient 162, where this other patient has a discharge time prior to updated discharge time 1030′ of patient 162. Thus, resources of physical therapy/occupational therapy department 814 are better used service a patient that may be discharged, that servicing patient 162 who cannot be discharged. Accordingly, as shown in FIG. 10A, mobility test 948 is also delayed for patient 162, as shown as mobility test 948′.

Since patient 162 has an update discharge time 1030′, dashboards 156, 500, and 600 indicate that patient 162 is not ready for discharge, and doctor 160 thereby delays visiting patient 162 on rounds. For example, after viewing dashboard 156, doctor 160 may skip patient 162 while visiting other patients within ward 908. As time progresses, mobility test 948 and medication delivery 1012 are completed and dashboard 156, 500, 600 update to indicate that patient 162 is ready for discharge. In one embodiment, patient 162 is represented as a flashing green sphere (e.g., flashing green sphere 706(4)) within dashboard 156 and doctor 162, seeing this flashing green sphere within an already visited ward, returns to visit and discharges patient 162. Thus, system 100 provides timely discharge of patient 162 is from hospital 170, and patient flow through hospital 170 is optimal.

Further, using dashboard 600 on mobile device 150, the delay to discharge patient 162 is quickly brought to the attention of doctor 160, where doctor may quickly identify the cause of the delay, and may thus attempt to resolve any problem to expedite discharge. For example, pharmacy circle 604(2) would be shown furthest from center circle 602.

Ultimately, when patient 162 has met all discharge criteria 404, doctor 160 has the final decision as to whether patient 162 is ready to go home. For example, even though patient 162 has met all discharge criteria 404, during rounds, doctor 160 may detect hesitancy within the speech and behavior of patient 162 indicating that patient 162 may not be mentally ready to leaving hospital. Doctor 160 may later return to patient 162 to investigate these reasons, but does not discharge patient 162. Thus, patient 162 may stay in hospital for another day.

### Tracking for Other Aspects of Hospital Management

Although certain of the examples above highlight the importance of discharge of patient 162 from hospital 170, system 100 also operates to track movement of patient 162 within hospital 170 and scheduling of services (e.g., patient movement 930 by transport department 812, MRI 924 by radiology department 810) for patient 162. For example, movement of patient 162 from ER 902 to bed 914 may be optimized through use of system 100, particularly when all movements between ER 902 and hospital 170, and movements within hospital 170 are considered as a whole.

Hospital services 120 are not coordinated to optimize patient movement through the hospital. Services are often applied to patients on a first-come/first-serve basis, or are applied on an as needed basis. Thus, although such application of services may work for a particular department, they do not allow for cooperation between departments to expedite movement of the patient through the hospital.

Beneficially, system 100 compares patient needs across multiple services and prioritizes utilization of all hospital services to improve patient flow within, and discharge from, the hospital. Without system 100, movement of a patient within the hospital becomes sporadic because progress or lack of progress of the patient through one service disrupts progress of the patient through another service. Each department is typically unaware how changes to its schedule affect other departments. Often, scheduled transport of a patient within the hospital does not occur because staff is busy transporting another patient. For example, transport staff may be unaware that Mr. Smith is expected to be in the hospital for another two weeks, but since he is at the top of the list for transport, he is transported to a hospital service for a specific test. However, since they are already transporting Mr. Smith, the transport staff cannot take Mrs. Jones who needs only one more test before being discharged. Thus, since Mrs. Jones does not have test results, she cannot be discharged and spends another day within the hospital unnecessarily. System 100 resolves this problem by prioritizing transport and testing of Mrs. Jones over Mr. Smith to improve patient flow through the hospital.

Analytic Analysis of Patient Movement within Hospital

In one embodiment, system 100 collects scheduling and movement information of patients within hospital 170, and provides this information to analytic engine 124. Analytic engine 124 collects patient movement information from many different hospitals, and builds a model based upon this movement data to determine how flow of patients through the hospitals may be further improved. For example, where one particular hospital service (e.g., placing service 808 within hospital 170) is determined as causing disruption to flow of patients through the hospital, improvements may be sought to that service. For example, using the imperial data collected by system 100, analytic engine 124 may determine that, for each patient, overlapping operation of a first service with a second service by one hour improved flow of patients through the hospital by twenty percent. Using the imperial data collected by system 100, analytic engine 124 may further determine that, for each patient, overlapping operation of these services by two hours improves flow of patient through the hospital by another ten percent. These optimizations may be applied to these services by adjusting the scheduling defined by system 100, for example. Thus, the movement of patients in any area of the hospital is optimized based upon patient flow through the hospital as a whole. This is accomplished by providing communication between each group within the hospital such that they are no longer independent entities, but operate as part of the whole hospital.

### Virtual Rounding

System 100 allows doctor 160 to make virtual rounds within hospital 170. Viewing dashboard 156, doctor 160 may notice that one (or more) of his patients is not progressing through hospital services 120 as fast as expected. For example, dashboard 156 may shows a red sphere (e.g., red sphere 706(1)) that represent patient 162 who is not progressing as fast as expected towards discharge from hospital 170. Upon noticing this red sphere, doctor 160 may “zoom-in” (e.g., pinching on a touch screen, or clicking on a sphere, or selecting a zoom button on a desktop display) to a portion of hospital 170 containing the red sphere, to get a more detailed status within that portion of hospital 170. See for example FIGS. 7A-7C and the description above that illustrates viewing details of each patient.

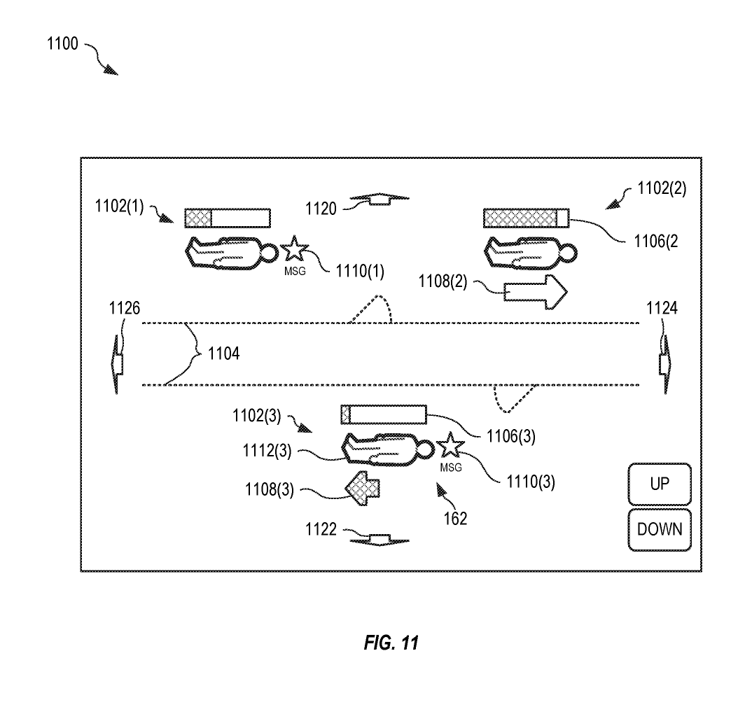

FIG. 11 shows one exemplary zoomed view 1100 of a portion of hospital 170 showing a status 1102 of each patient. Optionally, zoomed view 1100 may show structure 1104 (e.g., corridors) of hospital 170 to provide a better spatial reference as to the actual location of each displayed patient. Zoomed view 1100 includes direction arrows 1120, 1122, 1124, and 1126, for panning the view up, down, right, and left, respectively, to view other adjacent portions of hospital 170. Where hospital 170 has multiple floors, zoomed view 1100 may include an up button 1128 and a down button 1130 that select an adjacent floor of hospital 170 for view. Thus, doctor 160 may make “virtual rounds” using system 100.

As shown in FIG. 11, status 1102(3) represents patient 162 and shows a discharge readiness bar graph 1106(3), an overall progress arrow 1108(3), and a message indicator 1110(3). Doctor 160 may select the patient icon 1112(3) to open up a more detailed screen of the status of patient 162. In one embodiment, selection of patient icon 1112 may allow display of conventional electronic medical records of the corresponding patient. Discharge readiness bar graph 1106(3) is similar to bar graphs 506, 508 and shows the overall readiness to discharge of patient 162. Overall progress arrow 1108(3) provides an indication as to progress of the corresponding patient relative to expected progress. In the example of FIG. 11, progress arrow 1108(3) points to the left and is colored red, indicating that progress of patient 162 through hospital 170 is slower than expected. The size of progress arrow 1108 indicates the relative difference of the progress, where a larger arrow indicates a greater progress difference from expectation.

Message indicator 1110, when present, indicates that a communication related to the corresponding patient is waiting for doctor 160. Doctor 160 may wish to communicate with other care and service providers regarding progress of a particular patient. Ideally, these providers make rounds with doctor 162 such that the discussion may occur as doctor 162 focuses on the particular patient. However, coordination of providers with the doctor's rounds does not typically occur, and thus the doctor must remember to contact the provider after rounds are complete. Such communication typically involves leaving phone messages and getting phone messages in return since both the doctor and the providers are typically busy and not available to answer calls. Alternatively, where email is used, the email messages appear within an inbox of the recipient and may be easily missed or delayed. System 100 provides a simpler mechanism for allowing doctor 162 to communicate with the appropriate provider, and allows the provider to communicate with doctor 162, within the context of a particular patient—as if the providers and the doctor were rounding together. System 100 may connect with other communication systems to provide notification of waiting messages.

In one example of operation, doctor 160 selects message indicator 1110 to view or hear a waiting message, and doctor 160 may reply to that message using system 100. Doctor 162 may also generate a new message, in the context of the corresponding patient, by selecting message indicator 1110. Selecting message icon 1110 opens a new window that allows doctor 162 to select a recipient (e.g., a social worker, one hospital service 120) such that doctor 162 may communicate with the provider in the context of the associated patient to learn of reasons for delays, other problems and complications for example.

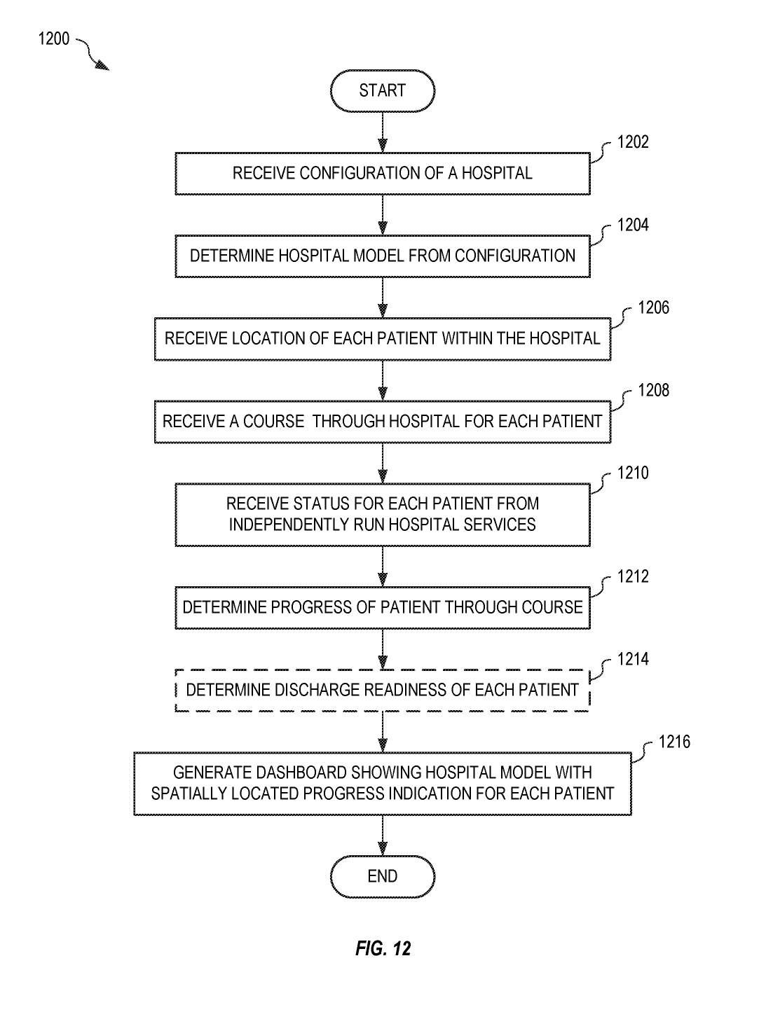

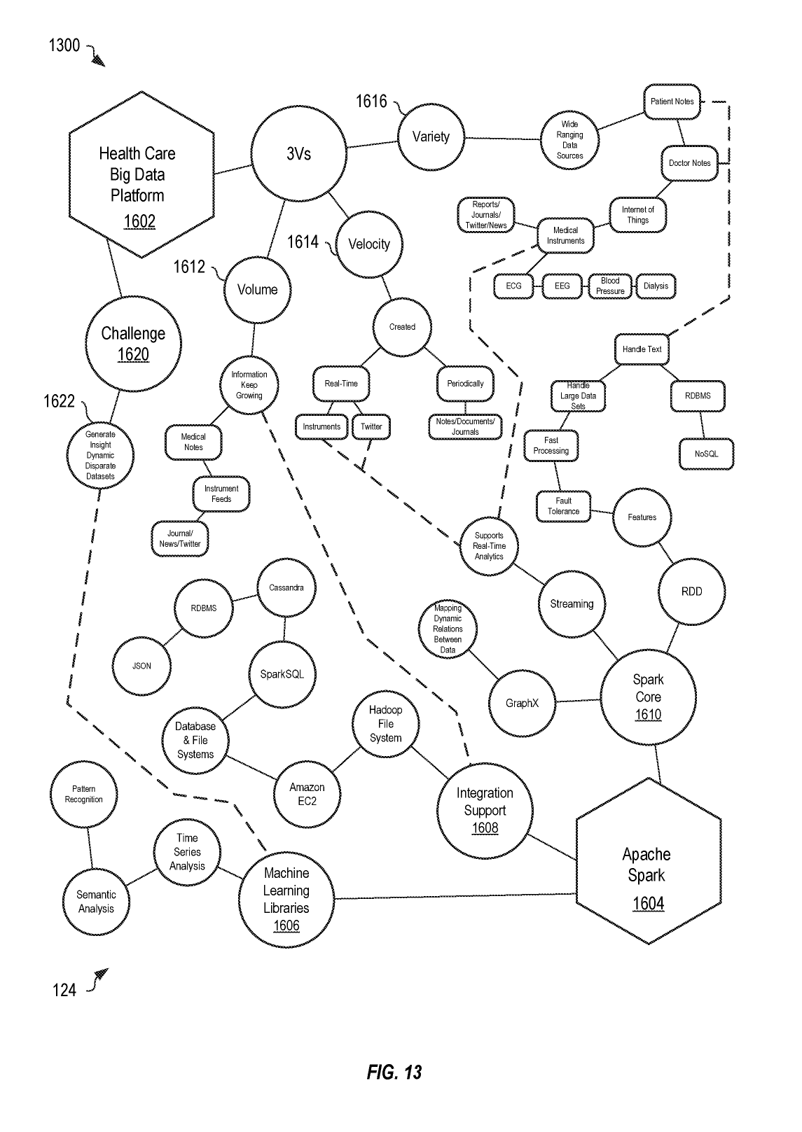

FIG. 12 is a flowchart illustrating one exemplary patient coordination method 1200. Method 1200 is for example implemented, at least in part, within server 102 of system 100 of FIG. 1.

In step 1202, method 1200 receives configuration of a hospital. In one example of step 1202, server 102 receives images of the hospital and a configuration defining a number of floors, a number of wards per floor, and a number of beds per ward. In step 1204, method 1200 determines a hospital model from the configuration. In one example of step 1204, patient status tracking algorithm 108 generates hospital model 112 from images 204, 208 and configuration 210. In step 1206, method 1200 receives location of each patient within the hospital. In one example of step 1206, algorithm 108 receives location of patient 162 as within bed 914 of ward 908. In step 1208, method 1200 receives a course through hospital for each patient. In one example of step 1208, algorithm 108 receives patient course 109 for patient 162. In step 1210, method 1200 receives a status for each patient from independently run hospital services, sensors or other data generating and providing means. In one example of step 1210, algorithm 108 receives patient status and scheduling information from each of laboratory 802, pharmacy 804, physiotherapy department 806, placing service 808, radiology department 810, transport department 812, and physical therapy/occupational therapy department 814. In other examples of step 1210, status information for each patient is gathered from various sensors associated with the patient, or other data generating devices.

In step 1212, method 1200 determines progress of each patient through the corresponding course. In one example of step 1212, algorithm 108 determines progress of patient 162 through patient course 109.

Step 1214 is optional. If included, in step 1214, method 1200 determines discharge readiness of each patient. In one example of step 1214, algorithm 108 determines discharge requirements from course 109 and for each discharge requirement, determines completeness.

In step 1216, method 1200 generates a dashboard showing the hospital model with spatially located progress indication for each patient. In one example of step 1216, algorithm 108 generates dashboard 156.

### Cost Benefits

As shown in FIG. 1, server 102 may include a cost algorithm 114 that determines cost savings made by system 100 based upon successful optimization of patient flow through hospital 170. Cost algorithm 114 may also operate to identify and accumulate costs caused by delay within hospital services 120 to provide a focus for areas of improvement. In one example, patient 162 is admitted to hospital 170 with symptoms including back pain, fever, and poor urine flow. Tests indicate a high white count in a urine sample, which also contains bacteria. Doctor 160 therefore diagnoses patient 162 as having a urinary infection. Typically, treatment of urinary infections required four to five days in hospital. However, where the patient is quickly diagnosed and appropriate medication is applied, they may recover to a point where discharge from hospital is possible after three days. However, typical prior art hospital procedure may take two or three days to discharge the patient because the discharge actions are performed serially. Through use of system 100, however, awareness of discharge readiness of patient 162, and processing of discharge actions to meet discharge criteria in parallel, allows patient 162 to be discharged when ready, thereby saving significant cost as compared to the prior art. By monitoring time and cost of patient 162 through hospital 170, cost tracking algorithm 114 easily shows cost savings and improvements in efficiency.

### Example Implementation

FIG. 13 shows one exemplary framework 1300 for implementing analytic engine 124 of FIG. 1 using an Apache Spark platform, in an embodiment. Framework 1300 depicts health care big data's 3Vs and expands them with health care examples.

A healthcare big-data platform 1302 is shown at the top left of framework 1300 and a ‘generic’ Apache Spark 1304 is shown at the bottom right. Framework 1300 includes three main hubs: machine learning libraries 1306, integration support 1308 and Spark core 1310. These hubs translate each of the three goals of a big-data platform: volume 1312, velocity 1314, and variety 1316.

Volume 1312 represents a huge volume of data received in various forms such as medical notes, and instrument feeds, to name a few, often received in time series or as continuous feed, and other data sources. This received data is stored, normalized, harvested and eventually ingested using framework 1300. These requirements are translated using Integration Support 1308. In this example embodiment, a database of analytic engine 124 is primarily implemented using Cassandra and uses the Hadoop File System hosted on an Amazon EC2 Virtual instance. Cassandra allowing queries to be run using SparkSQL and also provides support with standard data transport protocols such as JSON as may be used to transport data in FIG. 1.

### Velocity

Healthcare big-data platform 1302 supports real time data, which may be periodic or asynchronous, and functionality for processing these types of data is realized by exploiting the real time processing framework of Apache Spark 1304. For example, real-time feeds from various medical instruments, such as ECG, EEG, Blood Pressure Monitors or Dialysis Machines, shown as transducers 231 of system 100 in FIG. 2 of Appendix A of U.S. Patent Application Ser. No. 62/194,945.

### Variety

Healthcare big-data platform 1302 supports data from disparate sources. These data are processed by translating them through various modules that connects with ‘core’ Apache Spark modules. One such example is patient notes that contain natural language phrases 602 as shown in FIG. 6 of Appendix A of U.S. Patent Application Ser. No. 62/194,945. These modules include text handler, query processor (e.g., see FIG. 7 of Appendix A of U.S. Patent Application Ser. No. 62/194,945) and NoSQL database support. Another example is Speech Processing and Analysis as shown in FIG. 5 of Appendix B of U.S. Patent Application Ser. No. 62/194,945. These are mapped using a Resilient Distributed Data Set framework as supported by Apache Spark 1304.

### Big Data Analytics

Machine Learning Library 1306 provides access to standard machine learning algorithms such as pattern recognition, time series analysis, and semantic analysis. These algorithms may be used to process data from transducers 231 of FIGS. 2 and 3 of Appendix A of U.S. Patent Application Ser. No. 62/194,945, big data 450 of FIG. 4 of Appendix B of U.S. Patent Application Ser. No. 62/194,945, and phrase extraction and concept recognition tool 702 of FIG. 7 of Appendix B of U.S. Patent Application Ser. No. 62/194,945, for example. Framework 1300 thereby implements intelligence of analytic engine 224 of FIGS. 2, 4 and 5 of Appendix A of U.S. Patent Application Ser. No. 62/194,945, healthcare analytic engine 124 of FIGS. 1, 2, and 3 of Appendix B of U.S. Patent Application Ser. No. 62/194,945, and analytic engine 124 of FIG. 1. This described functionality is implemented by framework 1300 to overcome one of the biggest challenges 1320, how to process and generate insight from multiple disparate data sources 1322 within Healthcare big data platform 1302.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. In particular, the following embodiments are specifically contemplated, as well as any combinations of such embodiments that are compatible with one another:

(A1) A patient coordination system, including: a processor; a memory communicatively coupled with the processor; an interface, communicatively coupled with the processor, capable of receiving status information of a plurality of patients of a hospital; and a patient status tracking algorithm, implemented as machine readable instructions stored within the memory and executed by the processor.

(A2) The patient coordination systems denoted above as (A1), the processor, when executing the patient status tracking algorithm, capable of receiving configuration of a hospital.

(A3) Either of the patient coordination systems denoted above as (A1) and (A2), the processor, when executing the patient status tracking algorithm, further capable of determining a hospital model based upon the configuration.

(A4) Any of the patient coordination systems denoted above as (A1) through (A3), the processor, when executing the patient status tracking algorithm, further capable of receiving location of each patient within the hospital.

(A5) Any of the patient coordination systems denoted above as (A1) through (A4), the processor, when executing the patient status tracking algorithm, further capable of receiving a course through hospital for each patient.

(A6) Any of the patient coordination systems denoted above as (A1) through (A5), the processor, when executing the patient status tracking algorithm, further capable of receiving status for each patient from independently run hospital services.

(A7) Any of the patient coordination systems denoted above as (A1) through (A6), the processor, when executing the patient status tracking algorithm, further capable of determining progress of each patient through the corresponding course.

(A8) Any of the patient coordination systems denoted above as (A1) through (A7), the processor, when executing the patient status tracking algorithm, further capable of generating a dashboard showing the hospital model with spatial indication of the progress for each patient.

(A9) Any of the patient coordination systems denoted above as (A1) through (A8), further comprising a digital device for receiving and displaying the dashboard display.

(A10) The patient coordination systems denoted above as (A9), the digital device being selected from the group including: a fixed terminal at patient bedside, a nursing station, a computer on wheels (COW), a tablet, a personal digital assistant, a smartwatch, and a smartphone.

(A11) Either of the patient coordination systems denoted above as (A9) or (A10), wherein the dashboard displays is viewed by one of a doctor, a nurse, a case manager, a pharmacist, a social worker, a physical and occupational therapist, a dietician, a hospital administrator, a chaplain, a counselor, an ethicist, and other patient related health care personnel.

(A12) Any of the patient coordination systems denoted above as (A1) through (A11), the processor, when executing the patient status tracking algorithm, further capable of determining discharge criteria for each patient.

(A13) Any of the patient coordination systems denoted above as (A1) through (A13), the processor, when executing the patient status tracking algorithm, further capable of determining discharge readiness of each patient based upon the status and the discharge criteria.

(A14) Any of the patient coordination systems denoted above as (A1) through (A13), the processor, when executing the patient status tracking algorithm, further capable of displaying the discharge readiness of each patient spatially within the dashboard.

(A15) The patient coordination systems denoted above as (A14), the processor, when executing the patient status tracking algorithm, further capable of automatically updating the discharge readiness within the dashboard as the status of the patient changes.

(A16) Any of the patient coordination systems denoted above as (A1) through (A15), the status information comprising one or more of (a) status of one or more of the actions, (b) test results for the patient, (c) rehab information for the patient, (d) prescription information for the patient, and (e) placement information for the patient.

(A17) Any of the patient coordination systems denoted above as (A14) through (A16), the processor, when executing the patient status tracking algorithm, further capable of updating a schedule of one or more of the hospital services to improve patient flow through the hospital based upon one or both of the status and the progress.

(A18) Any of the patient coordination systems denoted above as (A1) through (A17), the independently run hospital services comprising one or more of a laboratory, a pharmacy, a physiotherapy department, a placing service, a radiology department, a transport department, and an physical therapy/occupational therapy department.

(B1) A patient coordination method, including receiving, within a server, configuration of a hospital; and determining a hospital model based upon the configuration.

(B2) The patient coordination method denoted above as (B1), further including receiving location of each patient within the hospital.

(B3) Either of the patient coordination methods denoted above as (B1) and (B2), further including receiving a course through hospital for each patient.

(B4) Any of the patient coordination methods denoted above as (B1) through (B3), further including receiving status for each patient from independently run hospital services.

(B5) Any of the patient coordination methods denoted above as (B1) through (B4), further including determining progress of each patient through the corresponding course.

(B6) Any of the patient coordination methods denoted above as (B1) through (B5), further including generating a dashboard showing the hospital model with spatial indication of the progress for each patient.

(B7) Any of the patient coordination methods denoted above as (B1) through (B6), further including determining discharge criteria for each patient.

(B8) Any of the patient coordination methods denoted above as (B1) through (B7), further including determining discharge readiness of each patient based upon the status and the discharge criteria.

(B9) Any of the patient coordination methods denoted above as (B6) through (B8), further including displaying the discharge readiness of each patient spatially within the dashboard.

(B10) The patient coordination methods denoted above as (B9), further including automatically updating the discharge readiness within the dashboard as the status of the patient changes.

(B11) Any of the patient coordination methods denoted above as (B1) through (B10), the status information comprising one or more of (a) status of one or more of the actions, (b) test results for the patient, (c) rehab information for the patient, (d) prescription information for the patient, and (e) placement information for the patient, (f) end-of-life and hospice decisions and/or information, (h) ethics information and/or decisions and (i) economic information.

(B12) Any of the patient coordination methods denoted above as (B6) through (B11), further including displaying the dashboard on a digital device.

(B13) Any of the patient coordination methods denoted above as (B6) through (B12), wherein the digital device is a mobile device.

(B14) Any of the patient coordination methods denoted above as (B6) through (B13), further including displaying the dashboard on a mobile device for viewing by one of a doctor, a nurse, a case manager, a pharmacist, a social worker, a physical and occupational therapist, a dietician, a hospital administrator, a chaplain, a counselor, an ethicist, and other patient related health care personnel.

(B15) Any of the patient coordination methods denoted above as (B4) through (B14), further including updating a schedule of one or more of the hospital services to improve patient flow through the hospital based upon one or both of the status and the progress.

(B16) Any of the patient coordination methods denoted above as (B4) through (B15), the independently run hospital services comprising one or more of a laboratory, a pharmacy, a physiotherapy department, a placing service, a radiology department, a transport department, and an physical therapy/occupational therapy department.

What is claimed is:

1. A patient coordination system, comprising:
a processor;
a memory communicatively coupled with the processor;
an interface, communicatively coupled with the processor, capable of receiving status information of a plurality of patients of a hospital; and
a patient status tracking algorithm, implemented as machine readable instructions stored within the memory and executed by the processor, capable of:
receiving configuration of a hospital;
determining a hospital model based upon the configuration;
receiving location of each patient within the hospital;
receiving a course through hospital for each patient;
receiving status for each patient from at least one of independently run hospital services and sensors associated with the patient;
determining progress of each patient through the corresponding course; and
generating a dashboard showing the hospital model with spatial indication of the progress for each patient.

2. The patient coordination system of claim 1, further comprising a digital device for receiving and displaying the dashboard display.

3. The patient coordination system of claim 2, the digital device being selected from the group including: a fixed terminal at patient bedside, a nursing station, a computer on wheels (COW), a tablet, a personal digital assistant, a smartwatch, and a smartphone.

4. The patient coordination system of claim 2, wherein the dashboard displays is viewed by one of a doctor, a nurse, a case manager, a pharmacist, a social worker, a physical and occupational therapist, a dietician, a hospital administrator, a chaplain, a counselor, an ethicist, and other patient related health care personnel.

5. The patient coordination system of claim 1, the patient status tracking algorithm further capable of:
determining discharge criteria for each patient;
determining discharge readiness of each patient based upon the status and the discharge criteria; and
displaying the discharge readiness of each patient spatially within the dashboard.

6. The patient coordination system of claim 5, the patient status tracking algorithm further capable of automatically updating the discharge readiness within the dashboard as the status of the patient changes.

7. The patient coordination system of claim 1, the status information comprising one or more of (a) status of one or more of the actions, (b) test results for the patient, (c) rehab information for the patient, (d) prescription information for the patient, and (e) placement information for the patient.

8. The patient coordination system of claim 5, the patient status tracking algorithm further capable of updating a schedule of one or more of the hospital services to improve patient flow through the hospital based upon one or both of the status and the progress.

9. The patient coordination system of claim 5, the independently run hospital services comprising one or more of a laboratory, a pharmacy, a physiotherapy department, a placing service, a radiology department, a transport department, and an physical therapy/occupational therapy department.

10. A patient coordination method, comprising:
receiving, within a server, configuration of a hospital;
determine a hospital model based upon the configuration;
receiving location of each patient within the hospital;
receiving a course through hospital for each patient;
receiving status for each patient from independently run hospital services;
determining progress of each patient through the corresponding course; and
generating a dashboard showing the hospital model with spatial indication of the progress for each patient.

11. The patient coordination method of claim 10, further comprising:
determining discharge criteria for each patient;
determining discharge readiness of each patient based upon the status and the discharge criteria; and
displaying the discharge readiness of each patient spatially within the dashboard.

12. The patient coordination method of claim 11, automatically updating the discharge readiness within the dashboard as the status of the patient changes.

13. The patient coordination method of claim 10, the status information comprising one or more of (a) status of one or more of the actions, (b) test results for the patient, (c) rehab information for the patient, (d) prescription information for the patient, and (e) placement information for the patient, (0 end-of-life and hospice decisions and/or information, (h) ethics information and/or decisions and (i) economic information.

14. The patient coordination method of claim 10, further comprising displaying the dashboard on a digital device.

15. The patient coordination method of claim 14, wherein the digital device is a mobile device.

16. The patient coordination method of claim 10, further comprising displaying the dashboard on a mobile device for viewing by one of a doctor, a nurse, a case manager, a pharmacist, a social worker, a physical and occupational therapist, a dietician, a hospital administrator, a chaplain, a counselor, an ethicist, and other patient related health care personnel.

17. The patient coordination method of claim 10, further comprising updating a schedule of one or more of the hospital services to improve patient flow through the hospital based upon one or both of the status and the progress.

18. The patient coordination method of claim 10, the independently run hospital services comprising one or more of a laboratory, a pharmacy, a physiotherapy department, a placing service, a radiology department, a transport department, and an physical therapy/occupational therapy department.
